(19) United States
(12) Patent Application Publication
(10) Pub. No.: US 2004/0024231 A1
(43) Pub. Date: Feb. 5, 2004

(54) 17Alpha fluoroalkyl steroids, method for producing the same and pharmaceutical compositions containing said compounds

(76) Inventors: Sven Ring, Jena (DE); Guenter Kaufmann, Jena (DE); Ralf Wyrwa, Oelknitz (DE); Walter Elger, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/381,789

(22) PCT Filed: Sep. 28, 2001
(86) PCT No.: PCT/DE01/03732

(30) Foreign Application Priority Data
Sep. 29, 2000 (DE) 100 49 736.5

(51) Int. Cl. C07J 7/00

(52) U.S. Cl. 552/557

(57) ABSTRACT

The invention relates to 17α fluoroalkyl steroids of the general formula (I). STEROID (I), wherein R₃ represents a group of the formula CnFmHo, wherein n=1, 2, 3, 4, 5 or 6, m>1 and m+o=2n+1. The invention further relates to methods for producing the same and to compositions that contain said compounds. The inventive composition of the general formula (I) possess androgenic activity.

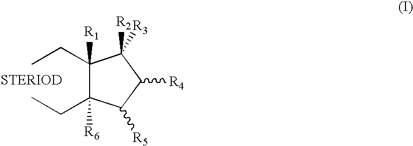

The invention relates to 17-methylene steroids, process for their production and pharmaceutical compositions that contain these compounds. The compounds according to the invention have androgenic activity.

17-Perfluoroalkylated compounds of the estrane- and 13-ethyl-gonane series are known. 17β-Hydroxy-17α-trifluoromethyl-estr-4-en-3-one, 17β-hydroxy-17α-trifluoromethyl-estra-4,9-dien-3-one and 17β-hydroxy-17α-trifluoromethyl-estra-4,9,11-trien-3-one, 13-ethyl-17β-hydroxy-17α-trifluoromethyl-gon-4-en-3-one, 13-ethyl-17β-hydroxy-17α-trifluoromethyl-gona-4,9-dien-3-one and 13-ethyl-17β-hydroxy-17α-trifluoromethyl-gona-4,9,11-trien-3-one were described in Sci. China, Ser. B: Chem. (1997), 40(3), 294-301, CN 94-11218 or Bioorg. Med. Chem. Lett. (1995), 5(17), 1899-1902. The compounds are to have gestagenic activity. 17β-Hydroxy-17α-tritluoromethyl-androst-4-en-3-one is described as an intermediate product in WO 9313122. 17-Pentafluoroethyl-alkylated steroids of the estrane or androstane series are not known to date.

This invention provides 17α-fluoroalkyl steroids of general formula (I)

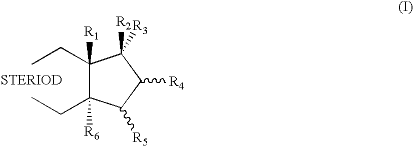

in which

R¹ stands for a C₁₋₄-alkyl group,

R² stands for a hydroxy group, a group OC(O)—R²⁰ or OR²¹, whereby R²⁰ and R²¹ mean a C₁₋₁₂-alkyl group, a C₃₋₈-cycloalkyl group, an aryl group or an aryl-C₁₋₄-alkyl group,

R³ stands for a radical of formula CnFmHo, whereby n=1, 2, 3, 4, 5 or 6, m>1 and m+o=2n+1,

R⁴ and R⁵ in each case stand for a hydrogen atom, together for a double bond or a methylene bridge,

R⁵ and R⁶ each stand for a hydrogen atom, together for a double bond or a methylene bridge,

STEROID stands for a steroidal ABC-ring system of partial formulas A, B, C, D, E and F:

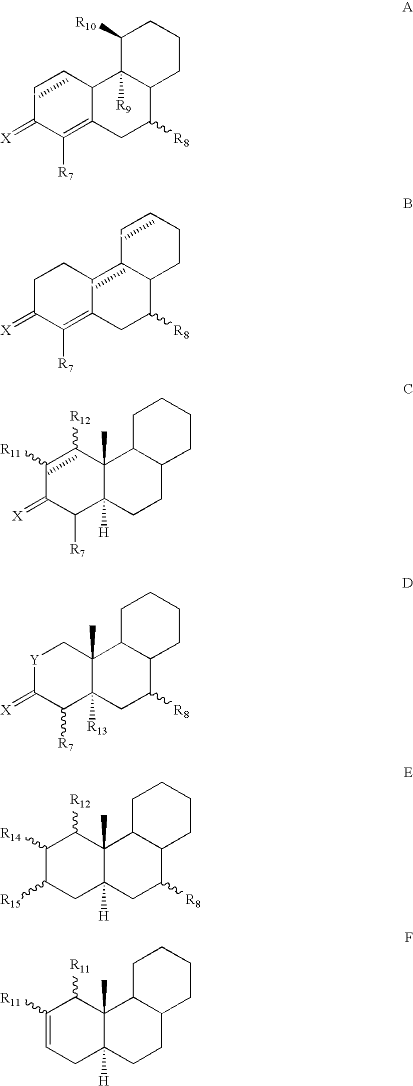

whereby an additional double bond can be found in A and C in 1,2-position, and one or two additional double bonds can be found in B in 8,9-position and 11,12-position,

R⁷ means a hydrogen atom, a halogen atom, a hydroxyl group or a trifluoromethyl group,

X means an oxygen atom, two hydrogen atoms or a hydroxyimino group,

R⁸ means a hydrogen atom, a methyl or ethyl group,

R⁹ means a hydrogen atom or a halogen atom or together with R¹⁰ stands for a double bond,

R¹⁰ means a hydrogen atom, a hydroxyl group, a methyl or ethyl group or together with R⁹ stands for a double bond,

R¹¹ means a hydrogen atom, a C₁₋₄-alkyl group, a nitrile group, a hydroxymethylene group or a formyl group,

R¹² means a hydrogen atom, a C₁₋₄-alkyl group or a nitrile group,

R¹¹ and R¹² in addition to the above-mentioned meanings together mean a methylene bridge,

R¹³ means a hydrogen atom or together with R⁷ means a double bond,

R¹⁴ and R¹⁵ together stand for a double bond, an oxirane ring, a thlirane ring, a [2,3c]oxadiazole ring, a [3,2c]isoxazole ring or a [3,2c]pyrazole ring,

Y stands for an oxygen or nitrogen atom,

and the wavy lines at R⁷, R⁸, R¹¹, R¹², R¹³, R¹⁴ and R¹⁵ mean that these substituents can be in α- or β-position and the following compounds are excluded:

17β-Hydroxy-17α-trifluoromethyl-androst-4-en-3-one

17β-Hydroxy-17α-trifluoromethyl-estr-4-en-3-one,

17β-Hydroxy-17α-trifluoromethyl-estra-4,9-dien-3-one,

17β-Hydroxy-17α-triiluoromnethyl-estra-4,9,11-trien-3-one,

13-Ethyl-17β-hydroxy-17α-tritluoromethyl-gon-4-en-3-one,

13-Ethyl-17β-hydroxy-17α-trifluoromethyl-gona-4,9-dien-3-one and

13-Ethyl-17β-hydroxy-17α-trifluoromethyl-gona-4,9,11-trien-3-one.

The compounds according to the invention have androgenic activity.

For the purposes of this invention, “C₁₋₄-alkyl group” is defined as a branched or straight-chain alkyl radical with 1 to 4 carbon atoms. As examples, a methyl, ethyl, n-propyl, i-propyl, n-butyl, i-butyl or tert.-butyl group can be mentioned.

For the purposes of this invention, “C₁₋₁₂-alkyl group” is defined as a branched or straight-chain alkyl radical with 1 to 12 carbon atoms. As examples, a methyl, ethyl, n-propyl, i-propyl, n-butyl, i-butyl, tert.-butyl, n-pentyl, i-pentyl, n-hexyl, 2-methylpentyl, 3-methylpentyl, 2,2-dimethylbutyl, 2,3-dimethylbutyl group, an octyl, nonyl, decyl or undecyl group can be mentioned.

According to the invention, the above-mentioned “C₃₋₈-cycloalkyl group” means a monocyclic or bicyclic group, such as, for example, a cyclopropyl, cyclobutyl, cyclopentyl or cyclohexyl group.

For the purposes of this invention, the term “aryl group” is defined as a substituted or unsubstituted aryl radical with 6 to 15 carbon atoms, for example a phenyl group, a substituted phenyl group, such as a halophenyl group, or a nitrophenyl group, or a naphthyl group.

For the purposes of this invention, the term “aryl-C₁₋₄-alkyl group” is defined as an alkyl radical that is substituted with an aryl radical, which together have 7 to 15 carbon atoms, whereby the aryl radical can carry other substituents, such as, for example, a halogen atom. Examples are a free or aromatic substituted benzyl group, such as a benzyl group or a halobenzyl group.

Within the scope of this invention, the term “halogen atom” is defined as a fluorine, chlorine, bromine or iodine atom.

The radical of formula CnFmHo, whereby n=1, 2, 3, 4, 5 or 6, m>1 and m+o=2n+1, can be a branched or straight-chain fluoroalkyl radical with 1 to 6 carbon atoms, whereby examples are a trifluoromethyl, pentafluoroethyl, heptafluoro-n-propyl group or a heptafluoro-iso-propyl group, whereby a trifluoromethyl group or a pentafluoroethyl group is preferred according to the invention.

If STEROID stands for a steroidal ring system of partial formula A, R₇ preferably means a hydrogen atom, a chlorine atom or a hydroxy group, R¹⁰ preferably means a hydrogen atom or a hydroxy group, and R⁹ preferably means a hydrogen atom or a fluorine atom.

If STEROID stands for a steroidal ring system of partial formula B, R₇ preferably means a hydrogen atom, a chlorine atom or a hydroxy group, and R₈ preferably means a hydrogen atom or a methyl group.

If STEROID stands for a steroidal ring system of partial formula C, R₇ preferably means a hydrogen atom, a chlorine atom or a hydroxy group, and R¹¹ preferably means a hydroxymethylene group or a formyl group or R¹¹ and R¹² together stand for a double bond.

If STEROID stands for a steroidal ring system of partial formula D, R₇ preferably means a hydrogen atom, a chlorine atom or a hydroxy group, R₈ preferably means a hydrogen atom or a methyl group, and R¹³ and R₇ together preferably mean a double bond, and Y preferably means an oxygen atom.

If STEROID stands for a seroidal ring system of partial formula E, R₈ preferably means a hydrogen atom or a methyl group, and R¹⁴ und R¹⁵ together preferably stand for a thiirane ring or a [3,2c]pyrazole ring.

If STEROID stands for a steroidal ring system of partial formula F, R¹¹ preferably means a C₁₋₄-alkyl group or a nitrile group.

R¹ preferably means a methyl group.

R² preferably means a hydroxy group, a formyloxy group, an acetyloxy group, a propionyloxy group, a butyryloxy group, a [(trans-4-butylcyclohexyl)carbonyl]oxy group, a phenylpropionyloxy group, an iso-butyryloxy group, a heptanyloxy group or an undecanyloxy group.

R³ preferably means a trifluoromethyl group or a pentafluoroethyl group.

Especially preferred 17α-fluoroalkyl steroids are cited below:

1) 17β-Hydroxy-17α-trifluoromethyl-7α-methyl-androst-4-en-3-one,

2) 17β,4-Dihydroxy-17α-trifluorometliyl-androst-4-en-3-one,

3) 17β-Hydroxy-17α-trifluoromethyl-4-chloro-androst-4-en-3-one,

4) 17β-Hydroxy-17α-trifluoromethyl-4-bromo-androst-4-en-3-one,

5) 17β-Hydroxy-17α,4-bis(trifluoromethyl)-androst-4-en-3-one,

6) 17β,11β-Dihydroxy-17α-trifluoromethyl-androst-4-en-3-one,

7) 17β,11β-Dihydroxy-17α-trifluoromethyl-9α-fluoro-androst-4-en-3-one,

8) 17β-Hydroxy-17α-trifluoromethyl-androsta-1,4-dien-3-one,

9) 17β-Hydroxy-17α-trifluoromethyl-4-chloro-androsta-1,4-dien-3-one,

10)17β,4-Dihydroxy-17α-trifluoromethyl-androsta-1,4-dien-3-one,

11)17β-Hydroxy-17α-trifluoromethyl-7α-methyl-androsta-1,4-dien-3-one,

12) 17β-Hydroxy-17α-trifluoromethyl-7α-methyl-4-chloro-androsta-1,4-dien-3-one,

13) 17β-Hydroxy-17α-pentafluoroethyl-androst-4-en-3-one,

14) 17β-Hydroxy-17α-pentafluoroethyl-7α-methyl-androst-4-en-3-one,

15) 17β,4-Dihydroxy-17α-pentafluoroethyl-androst-4-en-3-one,

16) 17β-Hydroxy-17α-pentafluoroethyl-4-chloro-androst-4-en-3-one,

17) 17β-Hydroxy-17α-pentafluoroethyl-4-bromo-androst-4-en-3-one.

18) 17β-Hydroxy-17α-pentafluoroethyl-4-trifluoromethyl-androst-4-en-3-one,

19) 17β,11β-Dihydroxy-17α-pentafluoroethyl-androst-4-en-3-one,

20) 17β,11β-Dihydroxy-17α-pentafluoroethyl-9α-fluoro-androst-4-en-3-one,

21) 17β-Hydroxy-17α-pentafluoroethyl-androsta-1,4-dien-3-one,

22) 17β-Hydroxy-17α-pentafluoroethyl-4-chloro-androsta-1,4-dien-3-one,

23) 17β3,4-Dihydroxy-17α-pentafluoroethyl-androsta-1,4-dien-3-one,

24) 17β-Hydroxy-17α-pentafluoroethyl-4-trifluoromethyl-androsta-1,4-dien-3-one,

25) 17β-Hydroxy-17α-pentafluoroethyl-7α-methyl-androsta-1,4-dien-3-one,

26) 17β-Hydroxy-17α-pentafluoroethyl-7α-methyl-4-chloro-androsta-1,4-dien-3-one,

27) 17β-Hydroxy-17α-trifluoromethyl-7α-methyl-estr-4-en-3-one,

28) 17β,4-Dihydroxy-17α-trifluoromethyl-estr-4-en-3-one,

29) 17β-Hydroxy-17α-trifluoromethyl-4-chloro-estr-4-en-3-one.

30) 17β-Hydroxy-17α-tritluoromethyl-4-bromo-estr-4-en-3-one,

31) 17β-Hydroxy-7α,4-bis(trifluoromethyl)-estr-4-en-3-one,

32) 17β-Hydroxy-17α-trifluoromethyl-7α-methyl-estra-4,9-dien-3-one,

33) 17β-Hydroxy-17α-trifluoromethyl-7α-methyl-estra-4,9,11-trien-3-one.

34) 17β-Hydroxy-17α-pentafluoroethyl-estr-4-en-3-one,

35) 17β-Hydroxy-17α-pentafluoroethyl-7α-methyl-estr-4-en-3-one,

36) 7β,4-Dihydroxy-17α-pentafluoroethyl-estr-4-en-3-one,

37) 17β-Hydroxy-17α-pentafluoroethyl-4-chloro-estr-4-en-3-one.

38) 17β-Hydroxy-17α-pentafluoroethyl-4-bromo-estr-4-en-3-one,

39) 17β-Hydroxy-17α-pentafluoroethyl-4-trifluoromethyl-estr-4-en-3-one,

40) 17β-Hydroxy-17α-pentafluoroethyl-estra-4,9-dien-3-one,

41) 17β-Hydroxy-17α-pentafluoroethyl-estra-4,9,11-trien-3-one,

42) 17β-Hydroxy-17α-pentafluoroethyl-7α-methyl-estra-4,9-dien-3-one,

43) 17β-Hydroxy-17α-pentafluoroethyl-7α-methyl-estra-4,9,11-trien-3-one,

44) 13-Ethyl-17β-hydroxy-17α-trifluoromethyl-4-chloro-gon-4-en-3-one,

45) 13-Ethyl-17β,4-dihydroxy-17α-trifluoromethyl-gon-4-en-3-one,

46) 13-Ethyl-17β-hydroxy-17α-trifluoromethyl-7α-methyl-gon-4-en-3-one,

47) 13-Ethyl-17β-hydroxy-17α-trifluoromethyl-7α-methyl-gona-4,9-dien-3-one,

48) 13-Ethyl-17β-hydroxy-17α-trifluoromethyl-7α-methyl-gona-4,9,11-trien-3-one,

49) 13-Ethyl-17β-hydroxy-17α-pentafluoroethyl-gon-4-en-3-one,

50) 13-Ethyl-17β-hydroxy-17α-pentafluoroethyl-7α-methyl-gon-4-en-3-one,

51) 13-Ethyl-17β,4-Dihydroxy-17α-pentatluoroethyl-gon-4-en-3-one,

52) 13-Ethyl-17β-hydroxy-17α-pentatluoroethyl-4-chloro-gon-4-en-3-one,

53) 13-Ethyl-17β-hydroxy-17α-pentafluoroethyl-4-bromo-gon-4-en-3-one,

54) 13-Ethyl-17β-hydroxy-17α-pentaluoroethyl-4-trifluoromethyl-gon-4-en-3-one,

55) 13-Ethyl-17β-hydroxy-17α-pentafluoroethyl-gona-4,9-dien-3-one,

56) 13-Ethyl-17β-hydroxy-17α-pentafluoroethyl-gona-4,9,11-tren-3-one,

57) 13-Ethyl-17β-hydroxy-17α-pentafluoroethyl-7α-methyl-gona-4,9-dien-3-one,

58) 13-Ethyl-17β-hydroxy-17α-pentafluoroethyl-7α-methyl-gona-4,9,11-trien-3-one,

59) 17β-Hydroxy-17α-trifluoromethyl-5α-androstan-3-one,

60) 17β-Hydroxy-17α-pentafluoroethyl-5α-androstan-3-one,

61) 17β-Hydroxy-17α-trifluoromethyl-7α-methyl-5α-androstan-3-one,

62) 17β-Hydroxy-17α-pentafluoroethyl-7α-methyl-5α-androstan-3-one,

63) 17β-Hydroxy-17α-trifluoromethyl-2-hydroxymethylene-5α-androstan-3-one,

64) 17β-Hydroxy-17α-pentafluoroethyl-2-hydroxymethylene-5α-androstan-3-one,

65) 17β-Hydroxy-17α-trnfluoromethyl-2α-methyl-5α-androstan-3-one,

66) 17β-Hydroxy-17α-pentafluoroethyl-2α-methyl-5α-androstan-3-one,

67) 17β-Hydroxy-17α-tnfluoromethyl-1α-methyl-5α-androstan-3-one,

68) 17β-Hydroxy-17α-pentafluoroethyl-1α-methyl-5α-androstan-3-one,

69) 17β-Hydroxy-17α-trifluoromethyl-5α-androst-2-ene,

70) 17β-Hydroxy-17α-pentafluoroethyl-5α-androst-2-ene,

71) 17β-Hydroxy-17α-trifluoromethyl-2-methyl-5α-androst-2-ene,

72) 17β-Hydroxy-17α-pentafluoroethyl-2-methyl-5α-androst-2-ene,

73) 17β-Hydroxy-17α-trifluoromethyl-2-cyano-5α-androst-2-ene,

74) 17β-Hydroxy-17α-pentafluoroethyl-2-cyano-5α-androst-2-ene,

75) 17β-Hydroxy-17α-trifluoromethyl-2-formyl-5α-androst-2-ene,

76) 17β-Hydroxy-17α-pentafluoroethyl-2-formyl-5α-androst-2-ene,

77) 17β-Hydroxy-17α-tnfluoromethyl-[2,3c]oxadiazole-5α-androstane,

78) 17β-Hydroxy-17α-pentafluoroethyl-[2,3c]oxadiazole-5α-androstane,

79) 17β-Hydroxy-17α-tlriluoromethyl-[3,2c]isoxazole-5α-androstane,

80) 17β-Hydroxy-17α-pentafluoroethyl-[3,2c]isoxazole-5 α-androstane,

81) 17β-Hydroxy-17α-trifluoromethyl-[3,2c]pyrazole-5α-androstane,

82) 17β-Hydroxy-17α-pentafiluoroethyl-[3,2c]pyrazole-5α-androstane,

83)17β-Hydroxy-17α-trifluoromethyl-2β,3β-epithio-5α-androstane,

84)17β-Hydroxy-17α-pentafluoroethyl-2β,3β-epithio-5α-androstane,

85)17β-Hydroxy-17α-trifluoromethyl-2α,3α-epithio-5α-androstane,

86) 17β-Hydroxy-17α-pentafluoroethyl-2α,3α-epithio-5α-androstane,

87)17β-Hydroxy-17α-trifluoromethyl-2-oxa-5α-androstan-3-one,

88)17β-Hydroxy-17α-pentafluoroethyl-2-oxa-5α-androstan-3-one,

89)17β-Hydroxy-17α-trifluoromethyl-5α-androst-1-en-3-one,

90)17β-Hydroxy-17α-pentafluoroethyl-5α-androst-1-en-3-one,

91) 17β-Hydroxy-17α-trifluoromethyl-1-methyl-5α-androst-1-en-3-one,

92)17β-Hydroxy-17α-pentafluoroethyl-1-methyl-5α-androst-1-en-3-one,

93)17β-Hydroxy-17α-trifluoromethyl-2-methyl-5α-androst-1-en-3-one and

94)17β-Hydroxy-17α-pentafluoroethyl-2-methyl-5α-androst-1-en-3-one.

Another subject of this invention is the process for the production of 17α-fluoroalkyl steroids of general formula (I), in which compounds of general formula (II)

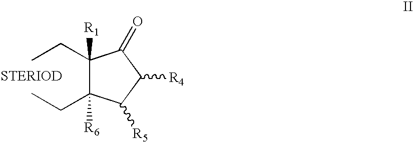

in which R¹, R⁴, R⁵, R⁶ and STEROID have the meaning given in claim 1, are reacted in the presence of fluoride with perfluoroalkyltrialkylsilanes, (Alk)₃SiCnFmHo, or with fluoroalkyl lithium, LiCnFmHo, or fluoroalkyl Grignard reagents, ZMgCnFmHo, whereby n=1, 2, 3, 4, 5 or 6, m>1 and m+o=2n, Z is a chlorine, bromine or iodine atom and Alk is a C₁₋₄-alkyl radical.

The introduction of the fluorinated 17α-alkyl chain can be carried out by adding (pertluoroalkyl)trimethylsilanes in the presence of fluoride (Rupperts Reagenz und seine Homologen [Rupperts Reagent and Its Homologs], J. Org. Chem. 1991, 56, 984-989) or by adding fluoroalkyl lithium or fluoroalk-yl Grignard reagents to the 17-oxo group of general formula II. The introduction of 17α-perfluoroalkyl chains can be carried out by adding perfluoroalkyl lithium to the 17-oxo group of general formula II (Tetr. Lett. 1985, 26, 5243; J. Org. Chem. 1987, 52, 2481).

Substituents R⁴, R⁵ and R⁶ that are mentioned in general formula II are introduced into the steroid-D ring advantageously before the introduction of the fluorinated 17α-alkyl chain according to the methods that are known to one skilled in the art.

For the production of compounds of general formula II with partial structures A to F, known steroid building blocks can be employed.

For example, the following steroid building blocks can be used:

For steroid building block A: Androst-4-ene-3,17-dione and dehydroepiandrosterone.

For steroid building block B: 19-Nortestosterone and 3,3-dimethoxy-estr-5(10)-17-one (DD 79-213049).

For steroid building block C, D or E: Epiandrosterone.

For steroid building block F: 5α-Androst-2-en-17-one from epiandrosterone (U.S. Pat. No. 3,098,851).

The functional groups contained in the partial structures of the starling materials for steroid building blocks A to F can optionally be protected according to the methods that are known to one skilled in the art.

Thus, keto groups in the starting materials of partial structures A to F can be protected as ketals or thioacetals according to methods that are known to one skilled in the art.

The introduction of substituents R⁷ to R¹⁵ in partial structures A to F can be carried out both before and after the incorporation of the fluorinated 17α-alkyl chain according to methods that are known to one skilled in the art.

The compounds according to the invention have androgenic activity, as the following receptor binding affinities to the androgen receptors illustrate.

| | RBA |
|---|---|
| Compound | % |
| Testosterone | 35 |
| R 1881 (Methyltrienolone) | 100 |
| 17β,4-Dihydroxy-17α-trifluoromethyl-estr-4-en-3-one | 15 |
| 17β-Hydroxy-17α-trifluoromethyl-7α-methyl-estr-4-en-3-one | 40 |
| 17β-Hydroxy-17α-trifluoromethyl-4-chloro-estr-4-en-3-one | 41 |
| 17β-Hydroxy-17α-trifluoromethyl-androst-4-en-3-one | 9.4 |
| 17β-Hydroxy-17α-trifluoromethyl-4-chloro-androst-4-en-3-one | 20 |
| 17β,11β-Dihydroxy-17α-trifluoromethyl-androst-4-en-3-one | 8.6 |
| 17β-Hydroxy-17α-trifluoromethyl-7α-methyl-androst-4-en-3-one | 30 |
| 17β-Hydroxy-17α-pentafluoroethyl-estr-4-en-3-one | 21 |
| 17β-Hydroxy-17α-pentafluoroethyl- 7α-methyl-estr-4-en-3-one | 34 |
| 17β,4-Dihydroxy-17α-pentafluoroethyl-estr-4-en-3-one | 20 |
| 17β-Hydroxy-17α-pentafluoroethyl-4-chloro-estr-4-en-3-one | 27 |

In the compounds of general formula (I) according to the invention, these test results open up a variety of possibilities for birth control in men, hormone replacement therapy (HRT) in men and women or the treatment of hormonally-induced diseases in men and women, such as, for example, endometriosis, breast cancer or hypooonadism.

Subjects of this invention are therefore also pharmaceutical compositions that contain at least one 17α-fluoroalkyl steroid of general formula (I), optionally together with pharmaceutically compatible adjuvants and vehicles.

These pharmaceutical compositions and pharmaceutical agents can be provided for oral, rectal, vaginal, subcutaneous, percutaneous, intravenous or intramuscular administration. In addition to commonly used vehicles and/or diluents, they contain at least one compound of general formula I.

The pharmaceutical agents of the invention are produced in a known way with commonly used solid or liquid vehicles or diluents and the commonly used pharmaceutical-technical adjuvants according to the desired type of administration with a suitable dosage. The preferred preparations consist in a dispensing form that is suitable for oral administration. Such dispensing forms are, for example, tablets, film tablets, coated tablets, capsules, pills, powders, solutions or suspensions or else depot forms.

Of course, parenteral preparations such as injection solutions are also considered. In addition, for example, suppositories and agents for vaginal application can also be mentioned as preparations.

Corresponding tablets can be obtained, for example, by mixing the active ingredient with known adjuvants, for example inert diluents such as dextrose, sugar, sorbitol, mannitol, polyvinyl pyrrolidone, explosives such as corn starch or alginic acid, binders such as starch or gelatin, lubricants such as magnesium stearate or talc and/or agents for achieving a depot effect such as carboxylpolymethylene, carboxylmethyl cellulose, cellulose acetate phthalate or polyvinyl acetate. The tablets can also consist of several layers.

Coated tablets can accordingly be produced by coating cores, which are produced analogously to the tablets, with agents that are commonly used in tablet coatings, for example polyvinyl pyrrolidone or shellac, gum arabic, talc, titanium oxide or sugar. In this case, the coated tablet shell can also consist of several layers, whereby the adjuvants that are mentioned above in the tablets can be used.

Solutions or suspensions with the compounds of general formula I according to the invention can contain in addition taste-improving agents such as saccharin, cyclamate or sugar, as well as, e.g., flavoring substances, such as vanilla or orange extract. In addition, they can contain suspending adjuvants such as sodium carboxymethyl cellulose or preservatives such as p-hydroxybenzoates.

Capsules that contain the compounds of general formula I can be produced, for example, by the compound(s) of general formula I being mixed with an inert vehicle such as lactose or sorbitol and encapsulated in gelatin capsules.

Suitable suppositories can be produced by, for example, mixing with vehicles that are provided for this purpose, such as neutral fats or polyethylene glycol or derivatives thereof.

The examples below explain this invention without the latter being limited thereto.

### EXAMPLE 1

### 17β-Hydroxy-17α-trifluoromethyl-androst-4-en-3-one

10 g of 3β-acetoxy-dehydroepiandrosterone is dissolved in 300 ml of THF and mixed with 0.5 g of tetrabutylammonium fluoride. While being stirred at room temperature, 15 ml of trifluoromethyltrimethylsilane is slowly added in drops, and it is stirred for 3 hours. Then, 200 ml of semiconcentrated sodium bicarbonate solution is added, and the THF is distilled off in a vacuum. The residue is extracted three times with 100 ml of ethyl acetate. The combined organic extracts are dried, concentrated by evaporation and chromatographed on silica gel. 12 g of 3β-acetoxy-17β-tiimethylsilyloxy-17α-trifluoromethyl-androst-5-ene is obtained.

12 g of 3β-acetoxy-17β-trimethylsilyloxy-17α-trifluoromethyl-androst-5-ene is dissolved in 100 ml of THF and mixed at room temperature with 20 ml of 30% hydrofluoric acid. After 3 hours, it is poured into 200 ml of 12% ammonia solution, extracted with 3×100 ml of ethyl acetate, the organic extracts are dried and concentrated by evaporation. 9 g of 3β-acetoxy-17β-hydroxy-17α-trifluoromethyl-androst-5-ene, which is dissolved in 300 ml of methanol and mixed with 6 g of potassium hydroxide, is obtained. After 30 minutes of stirring at room temperature, it is neutralized with 2N hydrochloric acid, and the methanol is drawn off in a vacuum. The residue is extracted with 4×100 ml of ethyl acetate, and the combined organic extracts are dried and concentrated by evaporation. 7.5 g of 3β,17β-dihydroxy-17α-trifluoromethyl-androst-5-ene, which is refluxed with 80 ml of cyclohexanone, 5 g of aluminum triisopropanolate and 250 ml of toluene for 3 hours, is obtained. It is allowed to cool, mixed with 200 ml of 2N sodium-potassium-tartrate solution, and the organic phase is separated and-extracted again with 2×100 ml of ethyl acetate. The combined organic extracts are concentrated by evaporation, the residue is purified by chromatographv and crystallized from methanol. 17β-Hydroxy-17α-trifluoromethyl-androst-4-en-3-one is obtained.

¹H-NMR (DMSO-D6): 0.89 (s, 3H, H-18), 1.15 (s, 3H, H-19), 5.62 (s, 1H, H-4)

¹⁹F-NMR: −75.3

### EXAMPLE 2

### 17β-Hydroxy-17α-trifluoromethyl-7α-methyl-androst-4-en-3-one

7 g of 17β-hydroxy-17α-trifluoromethyl-androst-4-en-3-one is refluxed with 8.5 g of chloranil in 200 ml of tert.-butanol for 30 minutes. It is allowed to cool and evaporated to the dry state. The residue is chromatographed on silica gel. For further purification, it is recrystallized from dichloromethaneihexane. 17β-Hydroxy-17α-trifluoromethyl-androsta-4,6-dien-3-one is obtained.

¹H-NMR: 1.04 (s, 3H, H-18), 1.13 (s, 3H, H-19), 5.69 (s, 1H, H-4), 6.11 (m, 2H, H-6, H-7)

¹⁹F-NMR: −75.3

80 ml of THF is added to a solution of methylmagnesium iodide (prepared from 2.5 of magnesium and 6.4 ml of methyl iodide in 80 ml of diethyl ether), it is cooled to −5° C., and 1 g of copper acetate-monohydrate, dissolved in 50 ml of THF, is added. It is cooled to −20° C., then a solution of 5 g of 17β-hydroxy-17α-trifluoromethyl-androsta-4,6-dien-3-one in 80 ml of THF is added in drops. After 2 hours, it is poured into ice water/2N sulfuric acid and extracted with 3×80 ml of ethyl acetate. The organic extract is dried and concentrated by evaporation. The residue is chromatographed on silica gel. For further purification, it is recrystallized from ethyl acetate. 17β-Hydroxy-17α-trifluoromethyt-7α-methyl-androst-4-en-3-one is obtained.

¹H-NMR: 0.77 (d, J=7 Hz, 3H, H-7-methyl), 0.99 (s, 3H, H-18), 1.20 (s, 3H, H-19), 5.73 (m, 1H, H-4)

¹⁹F-NMR: −75.3

### EXAMPLE 3

### 17β-Hydroxy-17α-trifluoromethyl-4-chloro-androst-4-en-3-one

Stage 1

17β-Hydroxy-17α-trifluoromethyl-4ξ,5ξ-epoxy-androstan-3-one

2 g of 17β-hydroxy-17α-trifluoromethyl-androst-4-en-3-one is dissolved in 120 ml of methanol and 70 ml of THF and mixed at 10° C. with 20 ml of hydrogen peroxide solution (35%). While being stirred, 5 ml of 10% sodium hydroxide solution is added, and it is stirred for 3 hours. The reaction solution is concentrated by evaporation to 50 ml then mixed with 50 ml of dichloromethane and 25 ml of water, and the organic phase is separated. It is washed with semiconcentrated thiosulfate solution, dried, and evaporated to the dry state. The residue that is obtained consists of a mixture of 4α,5α- or 4β,5β-epoxides and is used without further purification in the next stage.

Stage 2

17β-Hydroxy-17α-trifluoromethyl-4-chloro-androst-4-en-3-one

2 g of epoxide mixture (Stage 1) is dissolved in 200 ml of acetone and mixed at 5° C. with 12 ml of concentrated hydrochloric acid. After 2 hours, it is neutralized with soda solution, and the acetone is drawn off. The residue is extracted with dichloromethane. The organic extracts are dried and concentrated by evaporation. After crystallization from dichloromethane/hexane, 17β-hydroxy-17α-tritluoromethyl-4-chloro-androst-4-en-3-one is obtained.

¹H-NMR: 0.99 (s, 3H, H-18), 1.24 (s, 3H, H-19)

¹⁹F-NMR: −75.3

### EXAMPLE 4

### 17β,4-Dihydroxy-17α-trifluoromethyl-androst-4-en-3-one

2 g, of epoxide mixture (stage 1, production of 17β-hydroxy-17α-trifluoromethyl-4-chloro-androst-4-en-3-one) is dissolved in 20 ml of acetic acid, which contains 2% by volume of concentrated sulfuric acid. The solution is allowed to stand for 24 hours at 10° C. Then, it is mixed with 200 ml of ethyl acetate and neutralized with soda solution. The organic phase is dried and concentrated by evaporation. The residue is chromatographed on silica gel and crystallized from ethyl acetate/hexane.

¹H-NMR: 0.99 (s, 3H, H-18), 1.19 (s, 3H, H-19), 6.10 (s, 1H, 4-OH)

¹⁹F-NMR: −75.3

### EXAMPLE 5

### 17β-Hydroxy-17α-trifluoromethyl-androsta-1,4-dien-3-one

2 g of 17β-hydroxy-17α-trifluoromethyl-androst-4-en-3-one is stirTed with 1.8 g of DDQ in 60 ml of toluene for 60 hours at 85° C. Precipitate is filtered out, rewashed with toluene, and the filtrate is concentrated by evaporation. The residue is chromatographed on silica gel and recrystallized from ethyl acetate.

¹H-NMR: 1.02 (s, 3H, H-18), 1.24 (s, 3H, H-19), 6.07 (m, 1H, H-4), 6.22 (dd, J=1.6, 10 Hz, 1H, H-2), 7.04 (d, J=10 Hz, 1H, H-1)

¹⁹F-NMR: −75.4

### EXAMPLE 6

### 17β-Hydroxy-17α-trifluoromethyl-4-chloro-androsta-1,4-dien-3-one

Production of 17β-hydroxy-17α-trfluoromethyl-4-chloro-androst-4-en-3-one analogously to the instructions for 17β-hydroxy-17α-trifluoromethyl-androsta-1,4-dien-3-one.

¹H-NMR: 1.02 (s, 3H, H-18), 1.31 (s, 3H, H-19), 6.36 (d, J=10 Hz, 1H, H-2), 7.07 (d, J=10 Hz, 1H, H-1)

¹⁹F-NMR: −75.8

### EXAMPLE 7

### 17β-Hydroxy-17α-trifluoromethyl-7α-methyl-ester-4-en-3-one

Stage 1

7α-Methyl-estr-4-ene-3,17-dione

10 g of 17β-hydroxy-7α-methyl-estr-4-en-3-one (production: Steroids 1963, 317) is dissolved in 200 ml of acetone and oxidized at −20° C. with 15 ml of 8N chromosulfuric acid. After the reaction is completed, 10 ml of methanol is added, and it is allowed to heat to room temperature. The solvents are drawn off in a vacuum, and the residue is mixed with 300 ml of water, whereby the product precipitates. It is suctioned off, and 6.5 g of 7α-methyl-estr-4-ene-3,17-dione is obtained.

Stage 2

3,3-Ethylenedithio-7α-methyl-estr-4-en-17-one

5 g of 7α-methyl-estr-4-ene-3,17-dione is dissolved in 50 ml of methanol and mixed with 3 ml of ethanedithiol. 1.5 ml of boron trifluoride-diethyl etherate is added, and it is stirred at room temperature for 2 hours, whereby the product crystallizes out. It is suctioned off, and 6 g of 3,3-ethylenedithio-7α-methyl-estr-4-en-17-one is obtained.

Stage 3

17β-Hydroxy-17α-trifluoromethyl-7α-methyl-estr-4-en-3-one

5 g of 3,3-ethylenedithio-7α-methyl-estr-4-en-17-one is mixed at room temperature in 150 ml of THF with 0.25 g of tetrabutylammonium fluoride, and 8 ml of trifluoromethyltrimethylsilane is slowly added in drops. After 3 hours of stirring, 200 ml of semiconcentrated sodium bicarbonate solution is added, and the THF is distilled off in a vacuum. The residue is extracted three times with 100 ml of ethyl acetate. The combined organic extracts are dried, concentrated by evaporation and chromatographed on silica gel. 3 g of 17β-trimethylsilyloxy-17α-trifluoromethyl-3,3-ethylenedithio-7α-methyl-estr-4-ene is obtained. 3 g of 17β-trimethylsilyloxy-17α-trifluoromethyl-3,3-ethylenedithio-7α-methyl-estr-4-ene is dissolved in 40 ml of THF and mixed at room temperature with 5 ml of 30% hydrofluoric acid. After 3 hours, it is poured into 200 ml of 12% ammonia solution, extracted with 3×100 ml of ethyl acetate, the organic extracts are dried and concentrated by evaporation. The residue is taken up in 100 ml of 95% methanol, mixed with 9 ml of methyl iodide as well as 2.5 g of calcium carbonate and refluxed for 20 hours. After cooling, it is suctioned off, and the filtrate is evaporated to the dry state. The residue is chromatographed on silica gel and crystallized from dichloromethane/hexane. 17β-Hydroxy-17α-trifluoromethyl-7α-methyl-estr-4-en-3-one is obtained.

1H-NMR (D6-DMSO): 0.70 (d, J=7.7 Hz, 3H, 7-Methyl), 0.93 (s, 3H, H-18), 5.71 (s, 1H, H-4)

¹⁹F-NMR: −75.5

### EXAMPLE 8

### 17β-Hydroxy-17α-trifluoromethyl-4-chloro-estr-4-en-3-one and 17β,4-Dihydroxy-17α-trifluoromethyl-estr-4-en-3-on

Stage 1

17β-Hydroxy-17α-trifluoromethyl-estr-4-en-3-one

10 g of 3,3-dimethoxy-estr-5(10)-en-17-one is dissolved in 300 ml of THF and mixed with 0.5 g of tetrabutylammonium fluoride. While being stirred at room temperature, 15 ml of trifluoromethyltrimethylsilane is slowly added in drops, and it is stirred for 3 hours. Then, 200 ml of semiconcentrated sodium bicarbonate solution is added, and the THF is distilled off in a vacuum. The residue is extracted three times with 100 ml of ethyl acetate. The combined organic extracts are dried and concentrated by evaporation. 17β-Trimethylsilyloxy-17α-trifluoromethyl-3,3-dimethoxy-estr-5 (10)-ene is obtained.

12 g of 17β-trimethylsilyloxy-17α-trifluoromethyl-3,3-dimethoxy-estr-5(10)-ene is dissolved in 100 ml of THF and mixed at room temperature with 20 ml of 30% hydrofluoric acid. After 24 hours, it is poured into 200 ml of 12% ammonia solution, extracted with 3×100 ml of ethyl acetate, the organic extracts are dried and concentrated by evaporation. The residue is purified by cihromatooraphv on silica gel, and 17β-hydroxy-17α-trifluoromethyl-estr-4-en-3-one is obtained.

¹H-NMR(CDCl₃): 1.00 (s, 3H, H-18), 5.82 (s, 1H, H-4)

¹⁹F-NMR: −75.1

Stage 2

17β-Hydroxy-17α-trifluoromethyl-4ξ,5ξ-epoxy-estran-3-one

The production is carried out analogously to 17β-hydroxy-17α-trifluoromethyl-4ξ,5ξ-epoxy-androstan-3-one. The residue that is obtained consists of a mixture of 4α,5α- or 4β,5β-epoxides and is used without further purification in the next stage.

Stage 3

17β-Hydroxy-17α-trifluoromethyl-4-chloro-estr-4-en-3-one

The production is carried out from 17β-hydroxy-17α-trifluoromethyl-4ξ,5ξ-epoxy-estran-3-one analogously to 17β-hydroxy-17α-trifluoromethyl-4-chloro-androst-4-en-3-one.

¹H-NMR (CDCl₃): 1.00 (s, 3H, H-18)

¹⁹F-NMR: −75.3

Stage 4

17β,4-Dihydroxy-17α-trifluoromethyl-estr-4-en-3-one

The production is carried out from 17β-hydroxy-17α-trifluoromethyl-4ξ,5ξ-epoxy-estran-3-one analogously to 17β,4-dihydroxy-17α-trifluoromethyl-androst-4-en-3-one.

¹H-NMR (CDCl₃): 1.00 (s, 3H, H-18), 6.1 (s, 1H, 4-OH)

¹⁹F-NMR: −75.3

### EXAMPLE 9

### 17β-Hydroxy-17α-pentafluoroethyl-androst-4-en-3-one

20 g of 3,3-ethylenedithio-androst-4-en-17-one is suspended in 600 ml of diethyl ether and cooled to −78° C. while being stirred. 48 g of pentafluoroethyl iodide is added, then 76 ml of a 1.5 m solution of methyl lithium-lithium bromide complex in diethyl ether is slowly added in drops. It is stirred for 2 hours at −78° C. and then poured into 2 l of saturated sodium bicarbonate solution. It is extracted with ethyl acetate, dried and concentrated by evaporation.

The residue is taken up in 500 ml of 95% methanol, mixed with 72 ml of methyl iodide as well as 20 g of calcium carbonate and refluxed for 20 hours. After cooling, it is suctioned off, and the filtrate is evaporated to the dry state. The residue is chromatographed on silica gel and recrystallized from ethyl acetate. 17β-Hydroxy-17α-pentafluoroethyl-androst-4-en-3-one is obtained.

¹H-NMR (CDCl₃): 0.99 (s, 3H, H-18), 1.19 (s, 3H, H-19), 5.74 (s, 1H, H-4)

¹⁹F-NMR: −77.3 (3F, CF₃), −119 (2F, CF₂)

### EXAMPLE 10

### 17β-Hydroxy-17α-pentafluoroethyl-4-chloro-androst-4-en-3-one and 17β,4-Dihydroxy-17α-pentafluoroethyl-androst-4-en-3-one

Stage 1

17β-Hydroxy-17α-pentafluoroethyl-4ξ,5ξ-epoxy-androstan-3-one

The production is carried out analogously [from] 17β-hydroxy-17α-trifluoromethyl-4ξ,5ξ-epoxy-androstan-3-one. The residue that is obtained consists of a mixrure of 4α,5α- or 4β,5β-epoxides and is used without further purification in the next stage.

Stage 2

17β-Hydroxy-17α-pentafluoroethyl-4-chloro-androst-4-en-3-one

The production is carried out from 17β-hydroxy-17α-pentafluoroethyl-4ξ,5ξ-epoxy-androstan-3-one analogously to 17β-hydroxy-17α-trifluoromethyl-4-chloro-androst-4-en-3-one.

¹H-NMR (CDCl₃): 0.99 (s, 3H, H-18), 1.23 (s, 3H, H-19)

¹⁹F-NMR: −77.4 (3F, CF₃), −119.2 (2F, CF₂)

Stage 3

17β,4-Dihydroxy-17α-pentafluoroethyl-androst-4-en-3-one

The production is carried out from 17β-hydroxy-17α-pentafluoroethyl-4ξ,5ξ-epoxy-androstan-3-one analogously to 17β,4-dihydroxy-17α-trifluoromethyl-androst-4-en-3-one.

¹H-NMR (CDCl₃): 0.98 (s, 3H, H-18), 1.18 (s, 3H, H-19), 6.09 (s, 1H, 4-OH)

¹⁹F-NMR: −77.4 (3F, CF₃), −119.5 (2F, CF₂)

### EXAMPLE 11

### 17β-Hydroxy-17α-pentafluoroethyl-7 α-methyl-androst-4-en-3-one

Stage 1

17β-Hydroxy-17α-pentafluoroethyl-androst-4,6-dien-3-one

1 g of 17β-hydroxy-17α-pentafluoroethyl-androst-4-en-3-one is refluxed with 1.2 of chloranil in 50 ml of tert.-butanol for 30 minutes. It is allowed to cool and evaporated to the dry state. The residue is chromatographed on silica gel.

¹H-NMR (CDCl₃): 1.04 (s, 3H, H-18), 1.12 (s, 3H, H-19), 5.68 (s, 1H, H-4), 6.11 (m, 2H, H-6, H-7)

¹⁹F-NMR: −77.4 (3F, CF₃), −119.0 (2F, CF₂)

160 ml of THF is added to a solution of methylmagnesium iodide (prepared from 5 g of magnesium and 13 ml of methyl iodide in 150 ml of diethyl ether), it is cooled to −5° C., and 2 g of copper acetate-monohydrate, dissolved in 100 ml of THF, is added. It is cooled to −20° C., and then a solution of 10 g of 17β-hydroxy-17α-pentafluoroethyl-androsta-4,6-dien-3-one in 120 ml THF is added in drops. After 2 hours, it is poured into ice water/2N sulfuric acid and extracted with 3×80 ml of ethyl acetate. The organic extract is dried and concentrated by evaporation. The residue is chromatographed on silica gel. For further purification, it is recrystallized from ethyl acetate.

¹H-NMR (CDCl₃): 0.78 (d J=8 Hz, 3H, H-7Me), 1.01 (s, 3H, H-18), 1.21 (s, 3H, H-19), 5.74 (s, 1H, H-4)

¹⁹F-NMR: −77.4 (3F, CF3), −119.3 (2F, CF2)

### EXAMPLE 12

### 17β-Hydroxy-17α-pentafluoroethyl-estr-4-en-3-one

20 g of 3,3-dimethoxy-estr-5(10)-en-17-one is dissolved in 600 ml of diethyl ether and cooled to −78° C. while being stirred. 48 g of pentafluoroethyl iodide is added, then 76 ml of a 1.5 m solution of methyl lithium-lithium bromide complex in diethyl ether is slowly added in drops. It is stirred for 2 hours at −78° C. and then poured into 2 l of saturated sodium bicarbonate solution. It is extracted with ethyl acetate, dried and concentrated by evaporation.

The residue is chromatographed on silica gel and recrystallized from ethyl acetate. 17β-Hydroxy-17α-pentafluoroethyl-estr-4-en-3-one is obtained.

¹H-NMR (CDCl₃): 1.02 (s, 3H, H-18), 5.83 (s, 1H, H-4)

¹⁹F-NMR: −77.3 (3F, CF₃), −119.2 (2F, CF₂)

### EXAMPLE 13

### 17β-Hydroxy-17α-pentafluoroethyl-7α-methyl-estr-4-en-3-one

3,3-Ethylenedithio-7α-methyl-estr-4-en-17-one (see production of 17β-hydroxy-17α-trifluoromethyl-7α-methyl-estr-4-en-3-one) is reacted analogously to the above with pentafluoroethyl iodide/methyl lithium-lithium bromide complex. The crude product is taken up in 500 ml of 95% methanol, mixed with 72 ml of methyl iodide as well as 20 g of calcium carbonate and refluxed for 20 hours. After cooling, it is suctioned off, and the filtrate is evaporated to the dry state. The residue is chromatographed on silica gel and recrystallized from ethyl acetate. 17β-Hydroxy-17α-pentafluoroethyl-7α-methyl-estr-4-en-3-one is obtained.

¹H-NMR (CDCl₃): 0.77 (d, J=8 Hz, 3H, H-7Me), 1.02 (s, 3H, H-18), 5.84 (s, 1H, H-4)

¹⁹F-NMR: −77.3 (3F, CF₃), −119.2 (2F, CF₂)

### EXAMPLE 14

### 17β,4-Dihydroxy-17α-pentafluoroethyl-estr-4-en-3-one

Stage 1

17β-Hydroxy-17×-pentafluoroethyl-4ξ,5ξ-epoxy-estran-3-one

The production is carried out analogously to 17β-hydroxy-17α-trifluoromethyl-4ξ,5ξ-epoxy-androstan-3-one. The residue that is obtained consists of a mixture of 4α,5α- or 4β,5β-epoxides and is used without further purification in the next stage.

Stage 2

17β,4-Dihydroxy-17α-pentafluoroethyl-estr-4-en-3-one

The production is carried out from 17β-hydroxy-17α-pentafluoroethyl-4ξ,5ξ-epoxy-estran-3-one analogously to 17β,4-dihydroxy-17α-trifluoromethyl-androst-4-en-3-one.

¹H-NMR (CDCl₃): 1.00 (s, 3H, H-18), 6.10 (s, 1H, 4-OH)

¹⁹F-NMR: −77.3 (3F, CF₃), −119.2 (2F, CF₂)

### EXAMPLE 15

### 17β-Hydroxy-17α-pentafluoroethyl-4-chloro-estr-4-en-3-one

The production is carried out from 17β-hydroxy-17α-pentafluoroethyl-4ξ,5ξ-epoxy-estran-3-one analogously to 17β-hydroxy-17α-trifluoromethyl-4-chloro-androst-4-en-3-one.

¹H-NMR (CDCl₃) 1.01 (s, 3H, H-18)

¹⁹F-NMR: −77.3 (3F, CF₃), −119.2 (2F, CF₂)

### EXAMPLE 16

### 17β-Hydroxy-17α-trifluoromethyl-5α-androstan-3-one

Stage 1

3β-Acetoxy-17β-trimethylsilyloxy-17α-trifluoromethyl-5α-androstane

10 g of 3β-acetoxy-epiandrosterone is dissolved in 300 ml of THF and mixed with 0.5 g of tetrabutylammonium fluoride. While being stirred at room temperature, 15 ml of trifluoromethyltrimethytsilane is slowly added in drops, and it is stirred for 3 hours. Then, 200 ml of semiconcentrated sodium bicarbonate solution is added, and the THF is distilled off in a vacuum. The residue is extracted three times with 100 ml of ethyl acetate. The combined organic extracts are dried, concentrated by evaporation and chromatographed on silica gel. 9 g of 3β-acetoxy-17β-trimethylsilyloxy-17α-trifluoromethyl-5α-androstane is obtained.

Stage 2

3β-Acetoxy-17β-hydroxy-17α-trifluoromethyl-5 α-androstane

9 g of 3β-acetoxy-17β-trimethylsilyloxy-17α-trifluoromethyl-5α-androstane is dissolved in 100 ml of THF and mixed at room temperature with 20 ml of 30% hydrofluoric acid. After 3 hours, it is poured into 200 ml of 12% ammonia solution, extracted with 3×100 ml of ethyl acetate, the organic extracts are dried and concentrated by evaporation. 7 g of 3β-acetoxy-17β-hydroxy-17α-trifluoromethyl-5α-androstane is obtained.

Stage 3

3β,17β-Dihydroxy-17α-trifluoromethyl-5α-androstane

7 g of 3β-acetoxy-17β-hydroxy-17α-trifiuoromethyl-5α-androstane is dissolved in 300 ml of methanol and mixed with 6 g of potassium hydroxide. After 30 minutes of stirring at room temperature, it is neutralized with 2N hydrochloric acid, and the methanol is drawn off in a vacuum. The residue is extracted with 4×100 ml of ethyl acetate, and the combined organic extracts are dried and concentrated by evaporation. 4.5 g of 3β,17β-dihydroxy-17α-trifluoromethyl-5α-androstane is obtained.

Stage 4

17β-Hydroxy-17α-trifluoromethyl-5α-androstan-3-one

The product that is obtained in Stage 3 is oxidized in 50 ml of acetone with 9 ml of 8N chromosulfaric acid at 0° C. After the reaction is completed, 2 ml of methanol as well as 50 ml of water are added, and the acetone is drawn off in a vacuum, whereby the product precipitates. It is suctioned off and washed with water. For purification, it is chromatographed and crystallized from ethyl acetate. 17β-Hydroxy-17α-trifluoromethyl-5α-androstan-3-one is obtained.

¹H-NMR (CDCl₃): 0.96 (s, 3H, H-18), 1.02 (s, 3H, H-19)

¹⁹F-NMR: −75.4

### EXAMPLE 17

### 17β-Hydroxy-17α-pentafluoroethyl-5α-androstan-3-one

Stage 1

3β-Trimethylsilyloxy-5α-androstan-17-one

10 g of epiandrosterone is dissolved in 75 ml of DMF and 20 ml of pyridine and mixed with 10 ml of trimethylchlorosilane. After 3 hours, it is poured into 300 ml of saturated sodium bicarbonate solution. It is suctioned off, washed with water, and 15 g of 3β-trimethylsilyloxy-5α-androstan-17-one is obtained.

Stage 2

3β,17β-Dihydroxy-17α-pentafluoroethyl-5 α-androstane

15 g of 3β-trimethylsilyloxy-5 α-androstan-17-one is dissolved in 300 ml of diethyl ether and cooled to −78° C. 14 g of pentafluoroethyl iodide is added, and then 38 ml of a 1.5 m solution of methyl lithium-lithium bromide complex in diethyl ether is added in drops. It is allowed to stir for 1 hour and poured into 1 l of saturated sodium bicarbonate solution, extracted with ethyl acetate and concentrated by evaporation. The residue is taken up in 100 ml of THF and mixed with 8 g of tetrabutylammonium fluoride. After 30 minutes, 200 ml of saturated sodium bicarbonate solution is added, and the THF is drawn off in a vacuum, whereby the substance precipitates. It is suctioned off and washed with water. 13 g of 3β,17β-dihydroxy-17α-pentafluoroethyl-5α-androstane is obtained.

Stage 3

17β-Hydroxy-17α-pentafluoroethyl-5α-androstan-3-one

10 g of 3β,17β-dihydroxy-17α-pentafluoroethyl-5α-androstane is oxidized in 100 ml of acetone with 16 ml of 8N chromosulfuiic acid at 0° C. 3 ml of methanol and 100 ml of water are added, and the acetone is drawn off in a vacuum, whereby the product crystallizes out. It is suctioned off and washed with water. 17β-Hydroxy-17α-pentafluoroethyl-5α-androstan-3-one is obtained.

¹H-NMR (CDCl₃): 0.96 (s, 3H, H-18), 1.02 (s, 3H, H-19)

¹⁹F-NMR: −77.3 (3F, CF₃), −119.3 (2F, CF₂)

### EXAMPLE 18

### 17β-Hydroxy-17α-trifluoromethyl-2-hydroxymethylene-5α-androstan-3-one

4 g of 17β-hydroxy-17α-trifluoromethyl-5α-androstan-3-one is mixed in 150 ml of toluene with 3.2 g of sodium hydride and 8 ml of ethyl formate. After 24 hours, it is carefilly hydrolyzed with water. It is acidified with 5N hydrochloric acid, the organic phase is separated, dried and concentrated by evaporation. For purification, it is chromatographed on silica gel and crystallized from acetonelhexane. 17β-Hydroxy-17α-trifluoromethyl-2-hydroxymethylene-5α-androstan-3-one is obtained.

¹H-NMR (CDCl₃): 0.76 (s, 3H, H-18), 0.97 (s, 3H, H-19), 8.62 (m, 1H, H-2-CHO)

¹⁹F-NMR: −75.2

### EXAMPLE 19

### 17β-Hydroxy-17α-pentafluoroethyl-2-hydroxymethylene-5α-androstan-3-one

The compound is obtained analogously to Example 17 [?] from 17β-hydroxy-17α-pentafluoroethyl-5α-androstan-3-one.

¹H-NMR (CDCl₃): 0.77 (s, 3H, H-18), 0.96 (s, 3H, H-19), 8.62 (m, 1H, H-2-CHO)

¹⁹F-NMR: −77.3 (3F, CF₃), −119.2 (2F; CF₂)

### EXAMPLE 20

### 17β-Hydroxy-17α-trifluoromethyl-[3,2c] pyrazole-5α-androstane

1 g of 17β-hydroxy-17α-trifluoromethyl-2-hydroxymethylene-5α-androstan-3-one is refluxed in 50 ml of ethanol with the addition of 0.3 ml of hydrazine hydrate for 30 minutes. Then, it is concentrated by evaporation, precipitated with water and suctioned off. The product is purified by crystallization from tert.-butyl-methyl ether/hexane. 17β-Hydroxy-17α-trifluoromethyl-[3,2c]pyrazole-5α-androstane is obtained.

¹H-NMR (CDCl₃): 0.74 (s, 3H, H-18), 0.96 (s, 3H, H-19)

¹⁹F-NMR: −75.3

### EXAMPLE 21

### 17β-Hlydroxy-17α-pentafluoroethyl-[3,2c]pyrazole-5α-androstane

is obtained from 17β-hydroxy-17α-pentafluoroethyl-2-hydroxymethylene-5α-androstan-3-one analogously to Example 19.

¹H-NMR (CDCl₃): 0.74 (s, 3H, H-18), 0.96 (s, 3H, H-19)

¹⁹F-NMR: −77.3 (3F, CF₃), −119.3 (2F, CF₂)

### EXAMPLE 22

### 17β-Hydroxy-17α-trifluoromethyl-5α-androst-1-en-3-one

10 g of 17β-hydroxy-17α-trifluoromethyl-5α-androstan-3-one is mixed while being stirred in 200 ml of THF with 9 g of pyridinium-hydrobromide-perbromide. After 15 minutes, 250 ml of saturated sodium bicarbonate solution is added, extracted with chloroform, dried and concentrated by evaporation. The residue is refluxed with 10 g of lithium carbonate and 20 g of lithium bromide in 100 ml of DMF for 6 hours. It is allowed to cool, diluted with 500 ml of toluene, washed with water, dried and concentrated by evaporation. For purification, it is chromatographed on silica gel and recrystallized from ethyl acetate. 17β-Hydroxy-17α-trifluoromethyl-5α-androst-1-en-3-one is obtained.

¹H-NMR (CDCl₃): 0.98 (s, 3H, H-18), 1.02 (s, 3H, H-19), 5.85 (d, J=10 Hz, 1H, H-2), 7.12 (d, J=10 Hz, 1H, H-1)

¹⁹F-NMR: −75.3

### EXAMPLE 23

### 17β-Hydroxy-17α-pentafluoroethyl-5α-androst-1-en-3-one

is obtained from 17β-hydroxy-17α-pentafluoroethyl-5α-androstan-3-one analogously to Example 21.

¹H-NMR (CDCl₃): 0.98 (s, 3H, H-18), 1.02 (s, 3H, H-19), 5.85 (d, J=10 Hz, 1H, H-2), 7.12 (d, J=10 Hz, 1H, H-1)

¹⁹F-NMR: −77.3 (3F, CF₃), −119.2 (2F, CF₂)

### Example 24

### 17β-Hydroxy-17α-trifluoromethyl-2-oxa-5α-androstan-3-one

4 a of 17β-hydroxy-17α-trifluoromethyl-5α-androst-1-en-3-one is reacted in 200 ml of 90% acetic acid with 30 g of lead tetraacetate and 280 mg of osmium tetroxide. After 24 hours at room temperature, it is diluted with 500 ml of water and extracted three times with chloroform. The combined organic phases are alkalized with 2N sodium hydroxide solution and extracted three times with 200 ml of 2N sodium hydroxide solution. The combined aqueous phases are acidified with 5N hydrochloric acid and extracted three times with chloroform. The combined organic phases are dried and concentrated by evaporation.

The residue is dissolved in 80 ml of THF and 80 ml of methanol. While being stirred, a solution of 1 g of sodium bicarbonate in 75 ml of water and 4.2 g of sodium borohydride are added in succession. After 2 hours, it is acidified with concentrated hydrochloric acid, extracted with ethyl acetate, dried and concentrated by evaporation. For purification, it is chromatographed on silica gel and recrystallized from ethyl acetate. 17β-Hydroxy-17α-trifluoromethyl-2-oxa-5α-androstan-3-one is obtained.

¹H-NMR (CDCl₃): 0.94 (s, 3H, H-18), 1.00 (s, 3H, H-19), 2.22 (dd, J=19.1, 13.1 Hz, 1H, H-4), 2.53 (dd, J 18.7, 5.8 Hz, 1H, H-4), 3.92 (d, J=10 Hz, 1H, H-1), 4.21 (d, J=10 Hz, 1H, H-1)

¹⁹F-NMR: −75.4

### EXAMPLE 25

### 17β-Hydroxy-17α-pentafluoroethyl-2-oxa-5α-androstan-3-one

is obtained from 17β-hydroxy-17α-pentafluoroethyl-5α-androst-1-en-3-one analogously to Example 23.

¹H-NMR (CDCl₃) 0.94 (s, 3H, H-18), 1.00 (s, 3H, H-19), 2.22 (dd, J=19.1, 13.0 Hz, 1H, H-4), 2.53 (dd, J=19.1, 6.2 Hz, 1H, H-4), 3.93 (d, J=10 Hz, 1H, H-1), 4.22 (d, J=10 Hz, 1H, H-1)

¹⁹F-NMR: −77.3 (3F, CF₃), −119.2 (2F, CF₂)

### EXAMPLE 26

### 17β-Hydroxy-17α-trifluoromethyl-5α-androst-2-ene

Stage 1

17β-Trimethylsilyloxy-17α-trifluoromethyl-5α-androst-2-ene

10 g of 5α-androst-2-en-17-one (production according to U.S. Pat. No. 3,098,851) is dissolved in 300 ml THF and mixed with 0.5 g of tetrabutylammonium fluoride. While being stirred at room temperature, 15 ml of trifluoromethyltrimethylsilane is slowly added in drops, and it is stirred for 3 hours. Then, 200 ml of semiconcentrated sodium bicarbonate solution is added, and the THF is distilled off in a vacuum. The residue is extracted three times with 100 ml of ethyl acetate. The combined organic extracts are dried, concentrated by evaporation and chromatographed on silica gel. 10 g of 17β-trimethylsilyloxy-17α-trifluoromethyl-5α-androst-2-ene is obtained.

Stage 2

17β-Hydroxy-17α-trifluoromethyl-5α-androst-2-ene

10 g of 17β-trimethylsilyloxy-17α-tritluoromethyl-5α-androst-2-ene is dissolved in 100 ml of THF and mixed at room temperature with 20 ml of 30% hydrofluoric acid. After 3 hours, it is poured into 200 ml of 12% ammonia solution, extracted with 3×100 ml of ethyl acetate, the organic extracts are dried and concentrated by evaporation. After crystallization from methanol, 17β-hydroxy-17α-trifluoromethyl-5α-androst-2-ene is obtained.

¹H-NMR (CDCl₃): 0.76 (s, 3H, H-18), 0.93 (s, 3H, H-19), 5.60 (m, 2H, H-2,H-3)

¹⁹F-NMR: −75.1

### EXAMPLE 27

### 17β-Hydroxy-17α-pentafluoroethyl-5α-androst-2-ene

15 g of 5α-androst-2-en-17-one is dissolved in 300 ml of diethyl ether and cooled to −78° C. 14 g of pentafluoroethyl iodide is added, and then 38 ml of a 1.5 m solution of methyl lithium-lithium bromide complex in diethyl ether is added in drops. It is allowed to stir for 1 hour and poured into 1 l of saturated sodium bicarbonate solution, extracted with ethyl acetate and concentrated by evaporation. The residue is chromatographed on silica gel, and 17β-hydroxy-17α-pentafluoroethyl-5α-androst-2-ene is obtained.

¹H-NMR (CDCl₃): 0.76 (s, 3H, H-18), 0.95 (s, 3H, H-19), 5.62 (m, 2H, H-2, H-3)

¹⁹F-NMR: −77.5 (3F, CF₃), −119.3 (2f, CF₂)

1. 17α-fluoroalkyl steroids of general formula (1) 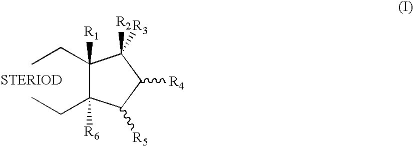
in which
R¹ stands for a C₁₋₄-alkyl group,
R² stands for a hydroxy group, a group OC(O)—R²⁰ or OR²¹, whereby R²⁰ and R²¹ mean a C₁₋₁₂-alkyl group, a C₃₋₈-cycloalkyl group, an aryl group or an aryl-C₁₋₄-alkyl group,
R³ stands for a radical of formula CnFmHo, whereby n=1, 2, 3, 4, 5 or 6, m>1 and m+o=2n+1,
R⁴ and R⁵ in each case stand for a hydrogen atom, together for a double bond or a methylene bridge,
R⁵ and R⁶ each stand for a hydrogen atom, together for a double bond or a methylene bridge,
STEROID stands for a steroidal ABC-ring system of partial formulas A, B, C, D, E and F: 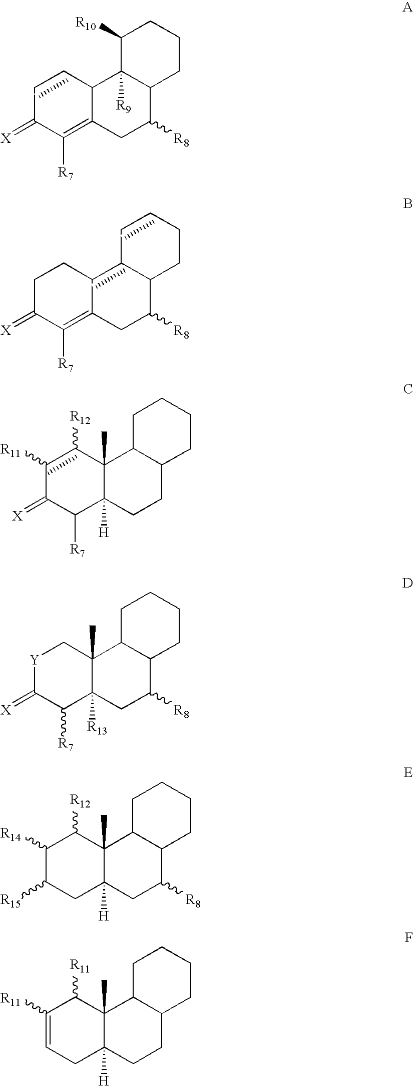
whereby an additional double bond can be found in A and C in 1,2-position, and one or two additional double bonds can be found in B in 8,9-position and 11,12-position,
R⁷ means a hydrogen atom, a halogen atom, a hydroxyl group or a trifluoromethyl group,
X means an oxygen atom, two hydrogen atoms or a hydroxyimino group,
R⁸ means a hydrogen atom, a methyl or ethyl group,
R⁹ means a hydrogen atom or a halogen atom or together with R¹⁰ stands for a double bond,
R¹⁰ means a hydrogen atom, a hydroxyl group, a methyl or ethyl group or together with R⁹ stands for a double bond,
R¹¹ means a hydrogen atom, a C₁₋₄-alkyl group, a nitrile group, a hydroxy-methylene group or a formyl group,
R¹² means a hydrogen atom, a C₁₋₄-alkyl group or a nitrile group,
R¹¹ and R¹², in addition to the above-mentioned meanings, together mean a methylene bridge,
R¹³ means a hydrogen atom or together with R⁷ means a double bond,
R¹⁴ and R¹⁵ together stand for a double bond, an oxirane ring, a thiirane ring, a [2,3c]oxadiazole ring, a [3,2c]isoxazole ring or a [3,2c]pyrazole ring,
Y stands for an oxygen or nitrogen atom,
and the wavy lines at R⁷, R⁸, R¹¹, R¹², R¹³, R¹⁴ and R¹⁵ mean that these substituents can be in α- or β-position, and the following compounds are excluded:
17β-Hydroxy-17α-trifluoromethyl-androst-4-en-3-one
17β-Hydroxy-17α-trifluoromethyl-estr-4-en-3-one,
17β-Hydroxy-17α-trifluoromethyl-estra-4,9-dien-3-one,
17β-Hydroxy-17α-trifluoromethyl-estra-4,9,11-trien-3-one,
13-Ethyl-17β-hydroxy-17α-trifluoromethyl-gon-4-en-3-one,
13-Ethyl-17β-hydroxy-17α-trifluoromethyl-gona-4,9-dien-3-one and
13-Ethyl-17β-hydroxy-17α-trifluoromethyl-gona-4,9,11-trien-3-one.

2. 17α-Fluoroalkyl steroids according to claim 1, whereby STEROID stands for a steroidal ring system of partial formula A.

3. 17α-Fluoroalkyl steroids according to claim 1, whereby STEROID stands for a steroidal ring system of partial formula B.

4. 17α-Fluoroalkyl steroids according to claim 1, whereby STEROID stands for a steroidal ring system of partial formula C.

5. 17α-Fluoroalkyl steroids according to claim 1, whereby STEROID stands for a steroidal ring system of partial formula D.

6. 17α-Fluoroalkyl steroids according to claim 1, wherebv STEROID stands for a steroidal ring system of partial formula E.

7. 17 α-Fluoroalkyl steroids according to claim 1, whereby STEROID stands for a steroidal ring system of partial formula F.

8. 17α-Fluoroalkyl steroids according to one of claims 1 to 7, whereby R¹ represents a methyl group.

9. 17α-Fluoroalkyl steroids according to one of claims 1 to 8, whereby R² means a hydroxy group, a formyloxy group, an acetyloxy group, a propionyloxy group, a butyryloxy group, a [(trans-4-butylcyclohexyl)carbonyl]oxy group, a phenylpropionyloxy group, an iso-butyryloxy group, a heptanyloxy group or an undecanyloxy group.

10. 17α-Fluoroalkyl steroids according to one of claims 1 to 9, whereby R³ represents a trifluoromethyl group or a pentafluoroethyl group.

11. 17α-Fluoroalkyl steroids according to one of claims 1 to 3 as well as 8 to 10, whereby R₈ is a methyl group.

12. 17α-Fluoroalkyl steroids according to one of claims 1 to 5 as well as 8 to 11, whereby R₇ represents a fluorine, chlorine or bromine atom, or a trifluoromethyl or hydroxy group.

13. 17α-Fluoroalkyl steroids according to one of claims 1, 2 as well as 8 to 11, whereby R¹⁰ represents a hydroxy group.

14. 17α-Fluoroalkyl steroids according to one of claims 1, 2 as well as 8 to 12, whereby R⁹ is a fluorine atom.

15. 17α-Fluoroalkyl steroids according to one of claims 1, 4, 7 and 12, whereby R¹¹ represents a hydroxymethylene group or a formyl group.

16. 17α-Fluoroalkyl steroids according to one of claims 1, 5, 11 and 12, whereby Y is an oxygen atom.

17. 17α-Fluoroalkyl steroids according to claim 1, namely
17β-Hydroxy-17α-trifluoromethyl-7α-methyl-androst-4-en-3-one,
17β,4-Dihydroxy-17α-trifluoromethyl-androst-4-en-3-one,
17β-Hydroxy-17α-trifluoromethyl-4-chloro-androst-4-en-3-one,
17β-Hydroxy-17α-trifluoromethyl-4-bromo-androst-4-en-3-one,
17β-Hydroxy-17α,4-bis(trifluoromethyl)-androst-4-en-3-one,
17β,11β-Dihydroxy-17α-trifluoromethyl-androst-4-en-3-one,
17β,11β-Dihydroxy-17α-tritluoromethyl-9 α-fluoro-androst-4-en-3-one,
17β-Hydroxy-17α-trifluoromethyl-androsta-1,4-dien-3-one,
17β-Hydroxy-17α-trifluoromethyl-4-chloro-androsta-1,4-dien-3-one,
17β,4-Dihydroxy-17α-trifluoromethyl-androsta-1,4-dien-3-one,
17β-Hydroxy-17a-trifluoromethyl-7α-methyl-androsta-1,4-dien-3-one,
17β-Hydroxy-17α-trifluoromethyl-7α-methyl-4-chloro-androsta-1,4-dien-3-one,
17β-Hydroxy-17α-pentailuoroethyl-androst-4-en-3-one,
17β-Hydroxy-17α-pentafluoroethyl-7α-methyl-androst-4-en-3-one,
17β,4-Dihydroxy-17α-pentafluoroethyl-androst-4-en-3-one,
17β-Hydroxy-17α-pentafluoroethyl-4-chloro-androst-4-en-3-one,
17β-Hydroxy-17α-pentafluoroethyl-4-bromo-androst-4-en-3-one,
17β-Hydroxy-17α-pentailuoroethyl-4-trifluoromethyl-androst-4-en-3-one,
17β,11β-Dihydroxy-17α-pentafluoroethyl-androst-4-en-3-one,
17β,11β-Dihydroxy-17α-pentafluoroethyl-9α-fluoro-androst-4-en-3-one,
17β-Hydroxy-17α-pentatluoroethyl-androsta-1,4-dien-3-one,
17β-Hydroxy-17α-pentafluoroethyl-4-chloro-androsta-1,4-dien-3-one,
17β,4-Dihydroxy-17α-pentafluoroethyl-androsta-1,4-dien-3-one,
17β-Hydroxy-17α-pentafluoroethyl-4-trifluoromethyl-androsta-1,4-dien-3-one,
17β-Hydroxy-17α-pentafluoroethyl-7α-methyl-androsta-1,4-dien-3-one,
17β-Hydroxy-17α-pentafluoroethyl-7α-methyl-4-chloro-androsta-1,4-dien-3-one,
17β-Hydroxy-17α-trifluoromethyl-7α-methyl-estr-4-en-3-one,
17β,4-Dihydroxy-17α-trifluoromethyl-estr-4-en-3-one,
17β-Hydroxy-17α-trifluoromethyl-4-chloro-estr-4-en-3-one,
17β-Hydroxy-17α-trifluoromethyl-4-bromo-estr-4-en-3-one,
17β-Hydroxy-17α,4-bis(trifluoromethyl)-estr-4-en-3-one,
17β-Hydroxy-17α-trifluoromethyl-7α-methyl-estra-4,9-dien-3-one,
17β-Hydroxy-17α-trifluoromethyl-7α-methyl-estra-4,9,11-trien-3-one,
17β-Hydroxy-17α-pentafluoroethyl-estr-4-en-3-one,
17β-Hydroxy-17α-pentafluoroethyl-7α-methyl-estr-4-en-3-one,
17β,4-Dihydroxy-17α-pentafluoroethyl-estr-4-en-3-one,
17β-Hydroxy-17α-pentafluoroethyl-4-chloro-estr-4-en-3-one,
17β-Hydroxy-17α-pentafluoroethyl-4-brorpo-estr-4-en-3-one,
17β-Hydroxy-17α-pentafluoroethyl-4-trifluoromethyl-estr-4-en-3-one,
17β-Hydroxy-17α-pentafluoroethyl-estra-4,9-dien-3-one,
17β-Hydroxy-17α-pentafluoroethyl-estra-4,9,11-trien-3-one,
17β-Hydroxy-17α-pentafluoroethyl-7α-methyl-estra-4,9-dien-3-one,
17β-Hydroxy-17α-pentafluoroethyl-7α-methyl-estra-4,9,11-trien-3-one,
13-Ethyl-17β-hydroxy-17α-trifluoromethyl-4-chloro-gon-4-en-3-one,
13-Ethyl-17β,4-hydroxy-17α-trifluoromethyl-gon-4-en-3-one
13-Ethyl-17β-hydroxy-17α-trifluoromethyl-7α-methyl-gon-4-en-3-one,
13-Ethyl-17β-hydroxy-17α-trifluoromethyl-7α-methyl-gona-4,9-dien-3-one,
13-Ethyl-17β-hydroxy-17α-trifluoromethyl-7α-methyl-gona-4,9, 11-trien-3-one,
13-Ethyl-17β-hydroxy-17α-pentafluoroethyl-gon-4-en-3-one,
13-Ethyl-17β-hydroxy-17α-pentafluoroethyl-7α-methyl-gon-4-en-3-one,
13-Ethyl-17β,4-dihydroxy-17α-pentafluoroethyl-gon-4-en-3-one,
13-Ethyl-17β-hydroxy-17α-pentafluoroethyl-4-chloro-gon-4-en-3-one,
13-Ethyl-17β-hydroxy-17α-pentafluoroethyl-4-bromo-gon-4-en-3-one,
13-Ethyl-17β-hydroxy-17α-pentafluoroethyl-4-trifluoromethyl-gon-4-en-3-one,
13-Ethyl-17β-hydroxy-17α-pentafluoroethyl-gona-4,9-dien-3-one,
13-Ethyl-17β-hydroxy-17α-pentafluoroethyl-gona-4,9,11-trien-3-one,
13-Ethyl-17β-hydroxy-17α-pentafluoroethyl-7α-methyl-gona-4,9-dien-3-one,
13-Ethyl-17β-hydroxy-17α-pentafluoroethyl-7α-methyl-gona-4,9,11-trien-3-one,
17β-Hydroxy-17α-tnifluoromethyl-5α-androstan-3-one,
17β-Hydroxy-17α-pentafluoroethyl-5α-androstan-3-one,
17β-Hydroxy-17α-trifluoromethyl-7α-methyl-5α-androstan-3-one,
17β-Hydroxy-17α-pentafluoroethyl-7α-methyl-5α-androstan-3-one,
17β-Hydroxy-17α-triuoromethyl-2-hydroxymethylene-5α-androstan-3-one,
17β-Hydroxy-17α-pentafluoroethyl-2-hydroxymethylene-5α-androstan-3-one,
17β-Hydroxy-17α-trifluoromethyl-2α-methyl-5α-androstan-3-one,
17β-Hydroxy-17α-pentafluoroethyl-2α-methyl-5α-androstan-3-one,
17β-Hydroxy-17α-trifluoromethyl-1α-methyl-5α-androstan-3-one,
17β-Hydroxy-17α-pentafluoroethyl-1α-methyl-5α-androstan-3-one,
17β-Hydroxy-17α-trifluorom ethyl-5α-androst-2-ene,
17β-Hydroxy-17α-pentafluoroethyl-5α-androst-2-ene,
17β-Hydroxy-17α-trifluoromethyl-2-methyl-5α-androst-2-ene,
17β-Hydroxy-17α-pentafluoroethyl-2-methyl-5α-androst-2-ene,
17β-Hydroxy-17α-trnfluoromethyl-2-cyano-5α-androst-2-ene,
17β-Hydroxy-17α-pentafluoroethyl-2-cyano-5α-androst-2-ene,
17β-Hydroxy-17α-trifluoromethyl-2-formyl-5α-androst-2-ene,
17β-Hydroxy-17α-pentafluoroethyl-2-formyl-5α-androst-2-ene,
17β-Hydroxy-17α-trifluoromethyl-[2,3 c]oxadiazole-5α-androstane,
17β-Hydroxy-17α-pentafluoroethyl-[2,3c]oxadiazole-5α-androstane,
17β-Hydroxy-17α-trifluoromethyl-[3,2c]isoxazote-5α-androstane,
17β-Hydroxy-17α-pentafluoroethyl-[3,2 c]isoxazole-5α-androstane,
17β-Hydroxy-17α-trifluoromethyl-[3,2c]pyrazole-5α-androstane,
17β-Hydroxy-17α-pentafluoroethyl-[3,2c]pyrazole-5α-androstane,
17β-Hydroxy-17α-trifluoromethyl-2β,3β-epithio-5α-androstane,
17β-Hydroxy-17α-pentafluoroethyl-2β,3β-epithio-5α-androstane,
17β-Hydroxy-17α-trifluoromethyl-2α,3α-epithio-5α-androstane,
17β-Hydroxy-17α-pentafluoroethyl-2α,3α-epithio-5αandrostane,
17β-Hydroxy-17α-trifluoromethyl-2-oxa-5α-androstan-3-one,
17β-Hydroxy-17α-pentafluoroethyl-2-oxa-5α-androstan-3-one,
17β-Hydroxy-17α-trifluoromethyl-5α-androst-1-en-3-one,
17β-Hydroxy-17α-pentafluoroethyl-5α-androst-1-en-3-one,
17β-Hydroxy-17α-trifluoromethyl-1-methyl-5α-androst-1-en-3-one,
17β-Hydroxy-17α-pentafluoroethyl-1-methyl-5α-androst-1-en-3-one,
17β-Hydroxy-17α-tritluoromethyl-2-methyl-5α-androst-1-en-3-one and
17β-Hydroxy-17α-pentafluoroethyl-2-methyl-5α-androst-1-en-3-one.

18. Process for the production of 17α-fluoroalkyl steroids of general formula (I) according to claim 1, characterized in that compounds of general formula (II) 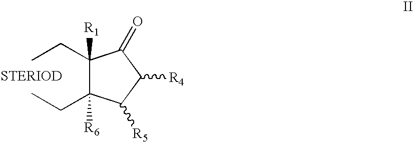
in which R¹, R⁴, R⁵, R⁶ and STEROID have the meanings given in claim 1, are reacted in the presence of fluoride with perfluoroalk-yltrialkylsilanes, (Alk)₃SiCnFmHo, or with fluoroalkyl lithium, LiCnFmHo, or fluoroalkyl Grignard reagents, ZMgCnFmHo, whereby n=1, 2, 4, 5 or 6, n>1 and m+o=2n, Z is a chlorine, bromine or iodine atom, and Alk is a C₁₋₄-alkyl radical.

19. Pharmaceutical compositions that contain at least one 17α-fluoroalkyl steroid of general formula (I) according to one of claims 1 to 17, optionally together with pharmaceutically compatible adjuvants and vehicles.

20. 17α-Fluoroalkyl steroids of formula (I) according to one of claims 1 to 17 for use as therapeutic active ingredients.

21. Use of 17α-fluoroalkyl steroids of general formula (I) according to one of claims 1 to 17 for birth control in men, for hormone replacement therapy (HRT) in men and women or for treatment of hormonally induced diseases in men and women, such as, for example, endometriosis, breast cancer or hypogonadism.
